(19) United States
(12) Patent Application Publication
(10) Pub. No.: US 2006/0161152 A1
(43) Pub. Date: Jul. 20, 2006

(54) Bone fixation systems and methods of assembling and/or installing the same

(75) Inventors: Michael D. Ensign, Salt Lake City, UT (US); David T. Hawkes, Pleasant Grove, UT (US)

Correspondence Address: RADER, FISHMAN & GRAUER PLLC, 10653 SOUTH RIVER FRONT PARKWAY, SUITE 150, SOUTH JORDAN, UT, 84095, US

(73) Assignee: Alphaspine, Inc., Sarasota, FL (US)

(21) Appl. No.: 11/258,393

(22) Filed: Oct. 24, 2005

Related U.S. Application Data
(60) Provisional application No. 60/622,107, filed on Oct. 25, 2004.

(51) Int. Cl. A61F 2/30

(52) U.S. Cl. 606/61

(57) ABSTRACT

A pedicle screw system includes a pedicle screw and a tulip assembly. A rod is inserted and fixed in the pedicle screw system to surgically connect vertebral bodies. The pedicle screw includes a head portion and a threaded shaft. In one embodiment, the tulip assembly includes a tulip body, a rod-support member, a collar member, a cap, and a fastening member. The tulip body has an upper portion and a lower portion, these portions being displaceable in opposite directions relative to an intermediate web. The tulip body can be intra-operatively coupled to the pedicle screw after the screw has been inserted into the bone. The cap engages with the tulip body to lock the tulip body onto the pedicle screw and thus fix the tulip body at a desired angle relative to the pedicle screw.

### CROSS-REFERENCE TO RELATED APPLICATION

This application claims the benefit under 35 U.S.C. § 119(e) of U.S. Provisional Patent Application No. 60/622,107 filed Oct. 25, 2004, where this provisional application is incorporated herein by reference in its entirety.

### BACKGROUND OF THE INVENTION

1. Field of the Invention

The present invention relates generally to bone fixation systems and methods of assembly, operation, and/or installation of these systems into cancellous and/or cortical bone.

2. Description of the Related Art

Various bone fixation systems for internally fixing, fusing, and/or otherwise supporting portions of a skeletal system for a human or other-type animal are known in the art. Bone fixation systems used along the spinal region of a patient are commonly referred to as a pedicle screw construct or a pedicle screw-coupling device because the pedicle screws are typically inserted and secured into the pedicles. Pedicles are generally understood to refer to the bone that bridges an anterior vertebral body to a lamina. U.S. Pat. No. 5,669,911 provides a detailed and thorough description of a spinal system including a discussion about the various bones and connective tissue associated therewith.

Pedicle screw constructs typically include a pedicle screw and a rod-coupling mechanism that are pre-operatively assembled. Some examples of pre-operatively assembled pedicle screw constructs are described in U.S. Published Patent Application Nos. 2005/0187548, 2005/0192571, and 2005/0216003. One drawback of pre-operatively assembled pedicle constructs is that the insertion of these constructs through the skin, muscle, and/or other tissue during surgery may cause damage and/or trauma to the tissue because the construct is larger and bulkier than the pedicle screw alone. In addition, the pre-operatively assembled pedicle constructs may be difficult to handle, maneuver, and to ultimately secure to the bone because the surgeon must direct the tool that drives the pedicle screw into the bone down through the rod-coupling mechanism to engage a driving portion of the pedicle screw. Further, the rod-coupling mechanism may be free to rotate relative to the pedicle screw, making it more difficult for the surgeon to guide and engage the tool with the pedicle screw.

### BRIEF SUMMARY OF THE INVENTION

The invention is related to systems and methods for achieving internal fixation of vertebral bodies.

In one aspect, a tulip assembly is coupleable to a pedicle screw having a head portion and a threaded shaft. The head portion is coupled to the threaded shaft. The tulip assembly includes a tulip body having an intermediate web, an upper portion positioned above the intermediate web, and a lower portion positioned below the intermediate web. The upper and lower portions are displaceable in substantially a radial direction relative to the intermediate web. The lower portion is radially expandable by an amount to intra-operatively receive the head portion of the pedicle screw, which may occur after the pedicle screw is secured into bone. A cap is engageable with the upper portion of the tulip body to lock the lower portion of the tulip body onto the head portion of the pedicle screw when the tulip body is at a desired angle relative to the pedicle screw.

In another aspect, a pedicle screw system includes a pedicle screw and a tulip assembly. The pedicle screw includes a head portion and a threaded shaft, where the head portion is coupled to the threaded shaft. The tulip assembly includes a tulip body and a cap. The tulip body has an intermediate web, an upper portion positioned above the intermediate web, and a lower portion positioned below the intermediate web. The upper and lower portions are displaceable in substantially a radial direction relative to the intermediate web. The lower portion is radially expandable by an amount to intra-operatively receive the head portion of the pedicle screw after the pedicle screw is secured into bone. The cap is engageable with the upper portion of the tulip body to lock the lower portion of the tulip body onto the head portion of the pedicle screw when the tulip body is at a desired angle relative to the pedicle screw and before the rod is locked into the tulip assembly.

In yet another aspect, a method is provided for installing a pedicle screw system into bone. The pedicle screw system includes a pedicle screw and a tulip assembly. The method begins by inserting the pedicle screw into the bone. Next, but not necessarily in the following sequence, the method includes coupling the tulip assembly to a head portion of the pedicle screw by radially, outwardly displacing a lower portion of a tulip body by an amount sufficient to receive the head portion of the pedicle screw. At least a section of a rod is placed onto a rod-support member that is positioned within the tulip body. The tulip assembly is fixed to the head portion of the pedicle screw by rotationally engaging a cap with the upper portion of the tulip body, the cap configured to cam the upper portion of the tulip body radially outward when rotated, and in turn, cause the lower portion of the tulip body to clamp onto the head portion of the pedicle screw. A fastening member is inserted into the tulip assembly to provide a downward force on at least a portion of the section of the rod to fixedly retain the same in the tulip assembly.

The foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages of the devices and/or processes described herein, as defined solely by the claims, will become apparent in the non-limiting detailed description set forth herein.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is an isometric view of a pedicle screw system, according to one illustrated embodiment.

FIG. 2A is a top plan view of a pedicle screw.

FIG. 2B is a partial cross-sectional elevational view of the pedicle screw of FIG. 2A seen along Section 2B-2B.

FIG. 3 is an exploded isometric view of a tulip assembly, according to one illustrated embodiment.

FIG. 4A is a top plan view of a tulip body from the tulip assembly of FIG. 3.

FIG. 4B is a partial cross-sectional elevational view of the tulip body of FIG. 4A seen along Section 4B-4B.

FIG. 5A is a top plan view of a collar member from the tulip assembly of FIG. 3.

FIG. 5B is a cross-sectional view of the collar member of FIG. 5A seen along Section 5B-5B.

FIG. 6A is a top, plan view of a rod-support member from the tulip assembly of FIG. 3.

FIG. 6B is a cross-sectional view of the rod-support member of FIG. 6A seen along Section 6B-6B.

FIG. 6C is a cross-sectional view of the rod-support member of FIG. 6A seen along Section 6C-6C.

FIG. 7A is a top plan view of a cap assembly from the tulip assembly of FIG. 3.

FIG. 7B is a cross-sectional view of the cap assembly of FIG. 7A seen along Section 7B-7B.

FIG. 7C is a partial, cross-sectional elevational view of the cap assembly of FIG. 7A seen along Section 7C-7C.

FIG. 8 shows a flow diagram of a method of assembling a pedicle screw system, according to one illustrated embodiment.

FIGS. 9A-9E cooperate with the flow diagram of FIG. 8 to show various stages of assembly.

FIG. 10 is a partial, cross-sectional view of the pedicle screw system of FIG. 1 showing the pedicle screw system in a fully assembled configuration.

### DETAILED DESCRIPTION OF THE INVENTION

In the following description, certain specific details are set forth in order to provide a thorough understanding of various embodiments of the present tools, assemblies, systems, and methods. However, one skilled in the relevant art will recognize that the assemblies, systems, and methods may be practiced without one or more of these specific details, or with other methods, components, materials, etc. In other instances, well-known structures associated with bone fixation systems and the assembly and/or installation thereof have not been shown or described in detail to avoid unnecessarily obscuring descriptions of the embodiments of the present assemblies, systems, and methods.

Unless the context requires otherwise, throughout the specification and claims which follow, the word “comprise” and variations thereof, such as “comprises” and “comprising,” are to be construed in an open, inclusive sense, that is, as “including, but not limited to.”Reference throughout this specification to “one embodiment” or “an embodiment” means that a particular feature, structure or characteristic described in connection with the embodiment is included in at least one embodiment of the present assemblies, devices, and systems. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the claimed invention.

Pedicle Screw System

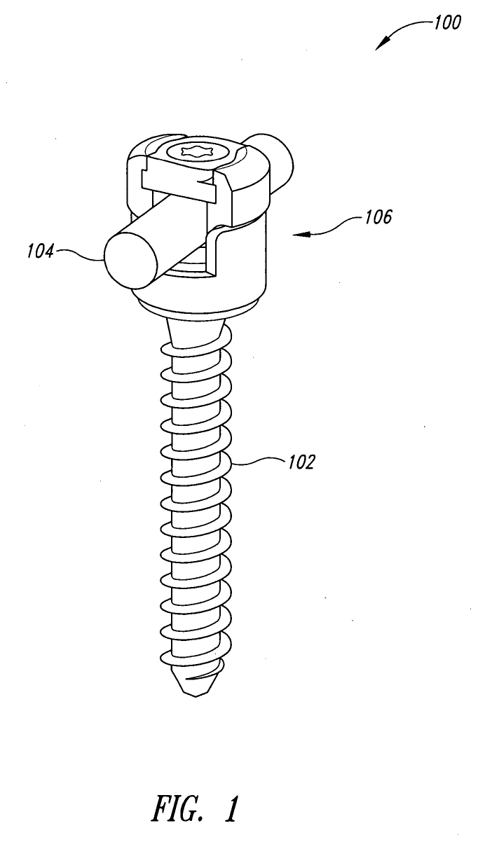

FIG. 1 generally shows a pedicle screw system 100 comprising a pedicle screw 102, a rod 104, and a coupling assembly 106, hereinafter referred to as a tulip assembly 106. During surgery, the tulip assembly 106 is coupled to the pedicle screw 102. A relative position of the tulip assembly 106 with respect to the pedicle screw 102 may be pre-operatively selected and then intra-operatively achieved. Once the relative position between the pedicle screw 102 and the tulip assembly 106 has been selected, the tulip assembly 106 is fixed or locked relative to the pedicle screw 102 before the rod is fixed or locked into the tulip assembly 106. It is understand that the relative, angular position of the tulip assembly 106 to the pedicle screw 102 may vary from one pedicle screw system 100 installation to another. In general, the relative position of the tulip assembly 106 to the pedicle screw 102 is selected to achieve a certain amount of spinal correction, which may involve compression, expansion, and/or rotation of at least a portion of a patient's spine.

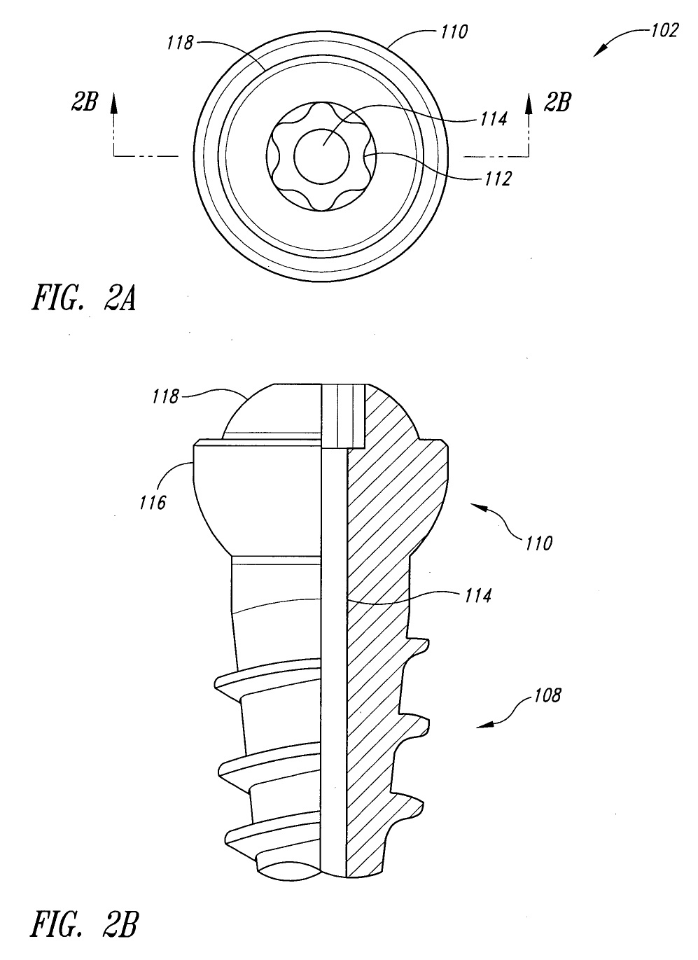

FIGS. 2A and 2B show the pedicle screw 102 having an elongated, threaded portion 108 and a head portion 110. Pedicle screws 102 are generally known in the art, but the head portions 110 may vary depending on what type of tulip assemblies 106 will be coupled to the pedicle screws 102. The head portion 110 of the pedicle screw 102 includes a driving feature 112, which is used for the initial insertion of the pedicle screw 102 into a pedicle, which is a part of a vertebra that connects the lamina with a vertebral body. In addition, the driving feature 112 may be used to adjust the pedicle screw 102 even after the tulip assembly 106 is coupled to the pedicle screw 102. In the illustrated embodiment, the head portion 110 of the pedicle screw 102 includes a dual diameter head comprising a greater diameter 116 and a lesser diameter 118.

In one embodiment, the pedicle screw 102 is cannulated, wherein a channel 114 extends through the entire length of the pedicle screw 102. The channel 114 allows the pedicle screw 102 to be maneuvered over and receive a Kirschner wire, commonly referred to as a K-wire. The K-wire is typically pre-positioned using imaging techniques, for example, fluoroscopy imaging.

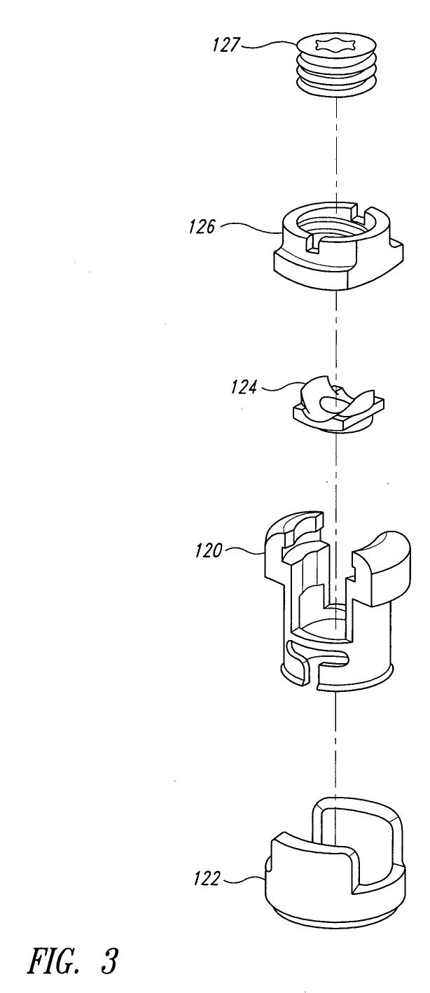

FIG. 3 shows the tulip assembly 106 comprising a coupling body 120 (hereinafter referred to as a tulip body 120), a collar member 122, a rod-support member 124, a cap 126, and a setscrew 127. The tulip assembly 106 and cap 126 may be assembled pre-operatively or, alternatively, may be assembled intra-operatively.

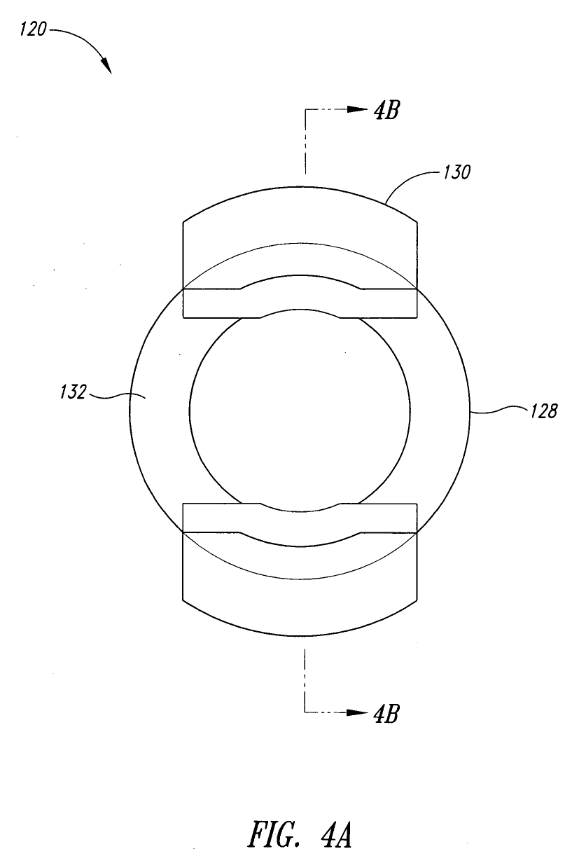

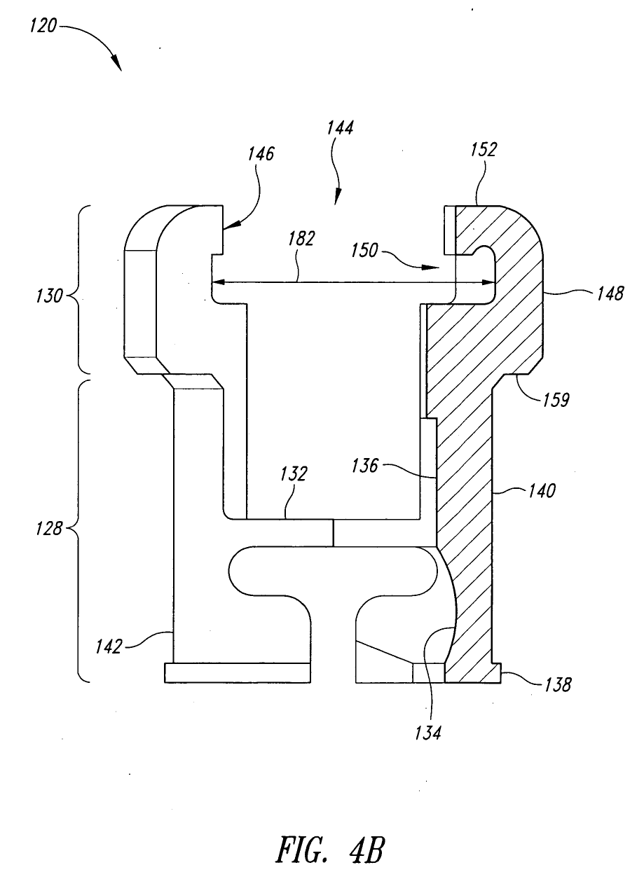

FIGS. 4A and 4B show the tulip body 120 having a lower portion 128 and an upper portion 130. The lower portion 128 includes a web 132, a spherical bore 134, a counterbored region 136, a radial flange 138, and first outer perimeter 140. The web 132 is positioned just above the spherical bore 134 and may operate as a fulcrum such that when the upper portion 130 is radially, outwardly displaced, or vice-versa, the lower portion 128 below the web 132 is simultaneously radially, inwardly displaced, or vice-versa, and the size and position of the web 132 remains relatively neutral to the applied displacement. Thus, the web 132 acts as a fulcrum by permitting the upper portions 130 to respond to radially inward or outward displacement of the lower portion 130 and vice-versa.

For example and as will be further described in the assembly methods below, the tulip body 120 is placed over the head portion 110 of the pedicle screw 102 by radially compressing (e.g., squeezing) the upper portion 130 of the tulip body 120 so that the spherical bore 134 opens by an amount sufficient to receive the head portion 110 of the pedicle screw 102. In one embodiment, the tulip body 120 is placed over the head portion 110 of the pedicle screw 102 intra-operatively (i.e., during surgery). Likewise, the lower portion 128, in particular a region 142 beneath the web 132, is compressible during pre-operative assembly of the tulip assembly 106. During pre-operative assembly, the region 142 is squeezed to cause the upper portion 130 to splay apart (i.e., open wider and/or expand) to allow the rod-support member 124 to be inserted into an opening 144 of the tulip body 120. In addition, squeezing the region 142 permits the collar member 122 to be moved over the radial flange 138 and encircle at least part of the lower portion 128 of the tulip body 120. It is understood that squeezing the region 142 may be achieved by applying an inward radial force on at least a portion of the first outer perimeter 140 that corresponds to the region 142. Additionally or alternatively, squeezing the region 142 may be achieved by applying an outward radial force to an inner surface 146 of the upper portion 130 of the tulip body 120 or by installing the cap 126 as will be described in more detail below.

The upper portion 130 includes a second outer perimeter 148 and a cap-mating groove 150. The second outer perimeter 148 is larger than the first outer perimeter 140 of the lower portion 128. The cap-mating groove 150 includes a lip 152. The cap-mating groove 150 is sized to receive the cap 126, wherein the lip 152 is arranged to retain the cap 126 as will be described in more detail below.

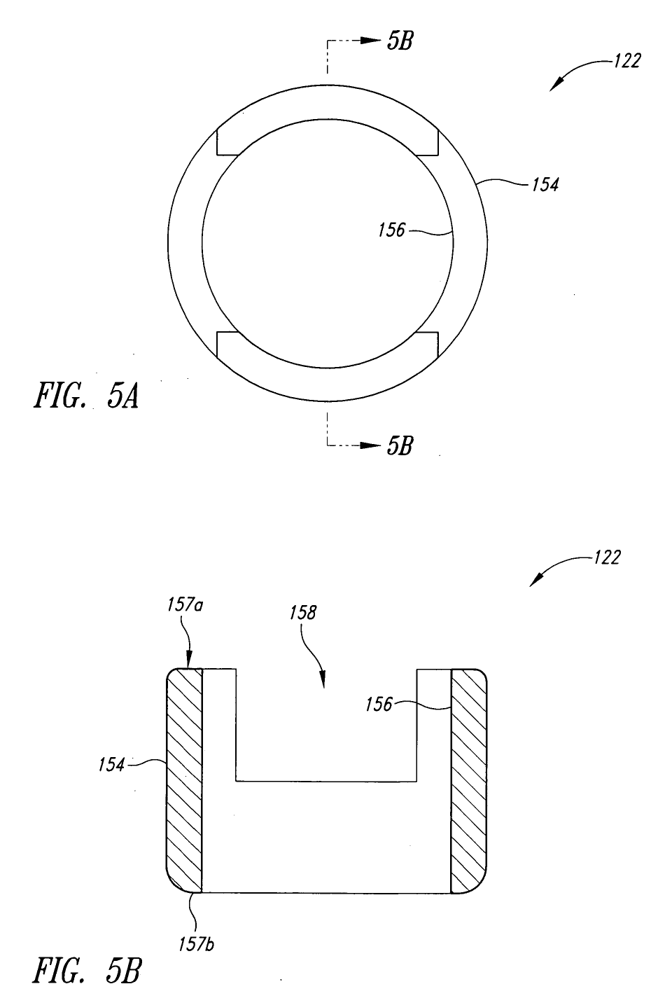

FIGS. 5A and 5B show the collar member 122 having an outer perimeter 154, an inner perimeter 156, an upper surface 157a, a lower surface 157b, and a cutout 158. The outer perimeter 154 is sized to be approximately the same as the second outer perimeter 148 of the upper portion 130 of the tulip body 120. In one embodiment, the collar member 122 is pre-operatively assembled with the tulip body 120. In particular, the inner perimeter 156 is sized to fit over and be slidable on the first outer perimeter 140 of the lower portion 128 of the tulip body 120, for example during pre-operative assembly. The upper surface 157a is configured to engage the shoulder 159 (FIG. 4B) of the tulip body 120 and the lower surface 157b is configured to engage the top portion of the radial flange 138 of the tulip body 120 during assembly therewith. The cutout 158 is wide enough and deep enough to receive at least a portion of the rod 104. Although the cutout 158 in the illustrated embodiment is relatively square or rectangular in shape, the cutout may take the form of a semi-circular arc, have more pronounced, radiused corners, or even have a parabolic shape, for example.

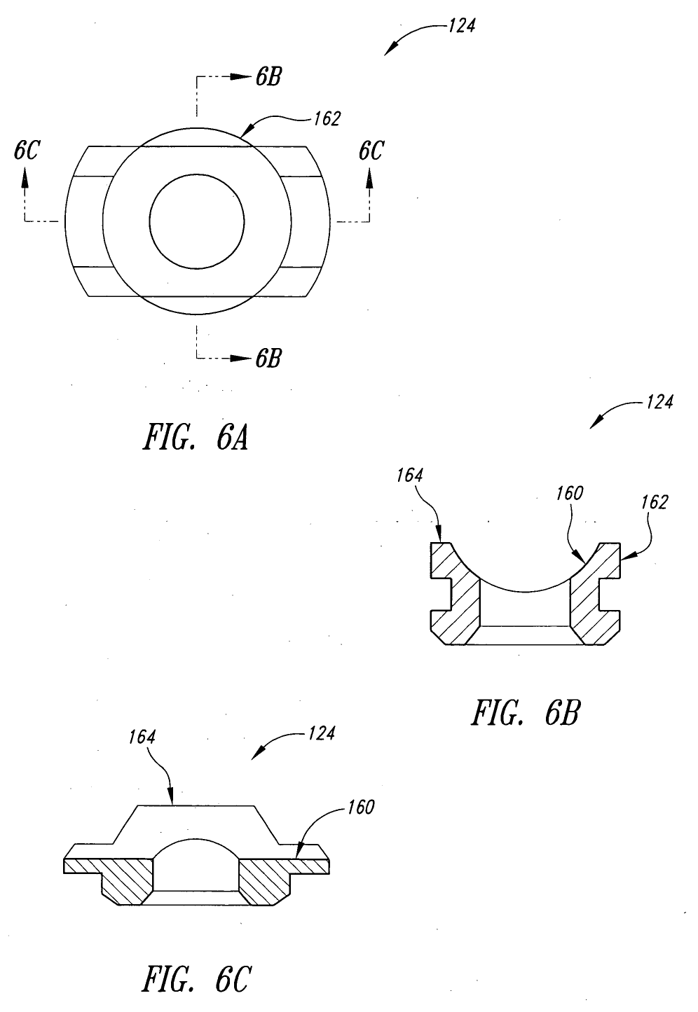

FIGS. 6A, 6B, and 6C show the rod-support member 124 having a rod-support surface 160, an outer surface 162, an upper surface 164, and an opening 166. The rod-support surface 160 is contoured to receive the rod 104. In one embodiment, a diameter of the rod-support surface 160 is contoured to achieve a tight fit with the rod 104, where the tight fit increases the contact stress and/or friction between the rod-support surface 160 and the rod 104.

The outer surface 162 is sized to fit through the opening 144 of the tulip body 120 and be placed in the counterbored region 136 of the tulip body 120 when the rod-support member 124 is pre-operatively assembled with the tulip body 120. The upper surface 164 engages a portion of the counterbored region 136 to retain the rod-support member 124 in the counterbored region 136 of the tulip body 120. The opening 166 of the rod-support member 124 permits access to the driving feature 112 of the pedicle screw 102. Accordingly, the opening 166 permits the adjustment of the pedicle screw 102 after the tulip assembly 106 has been coupled to the pedicle screw 102.

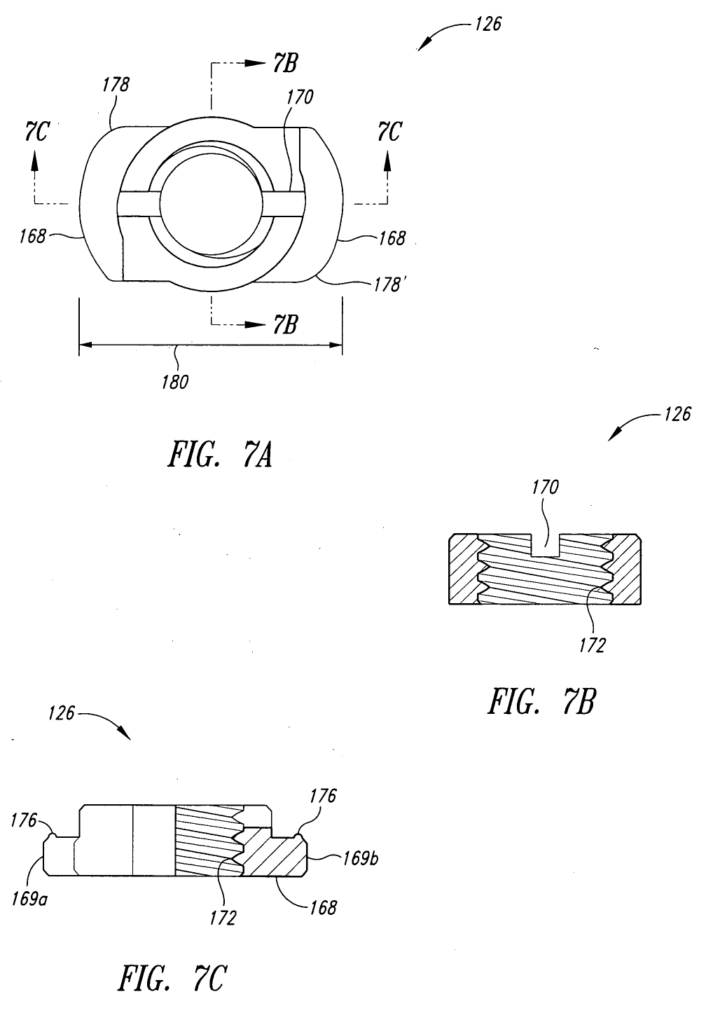

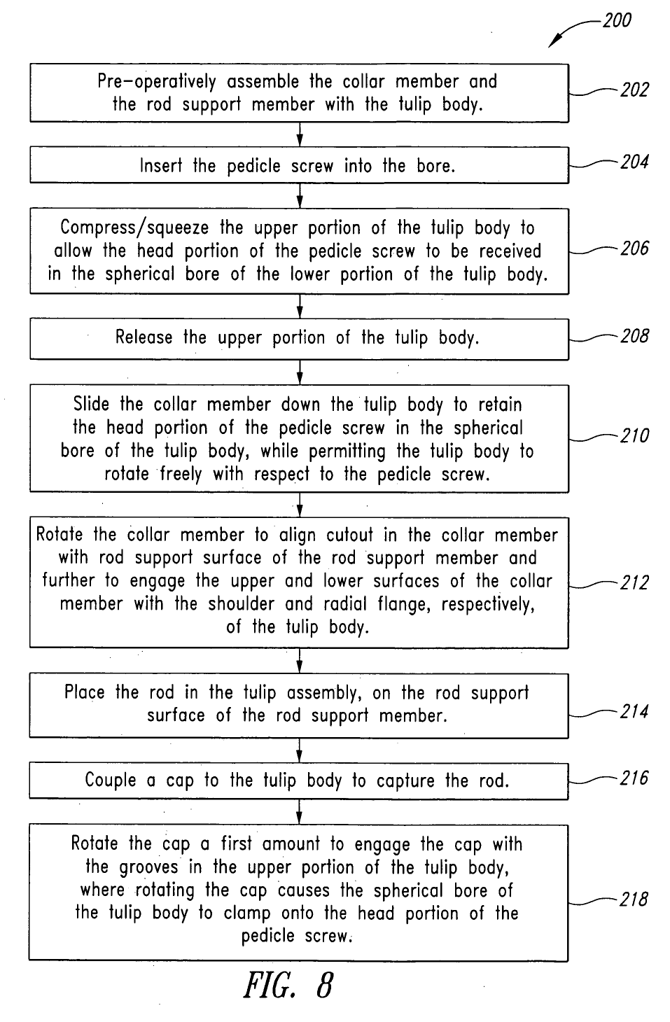

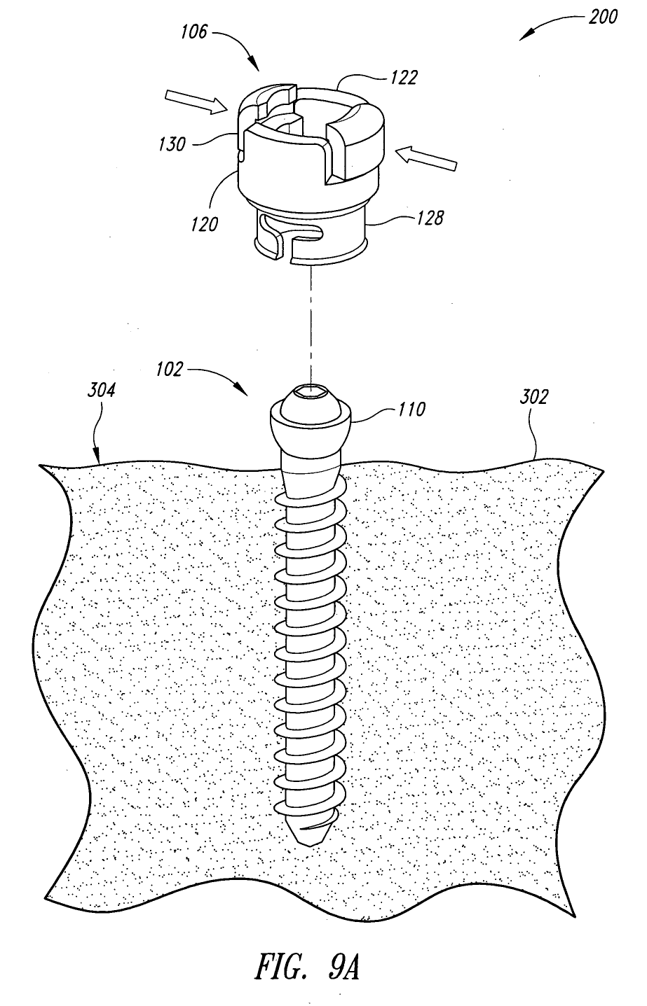

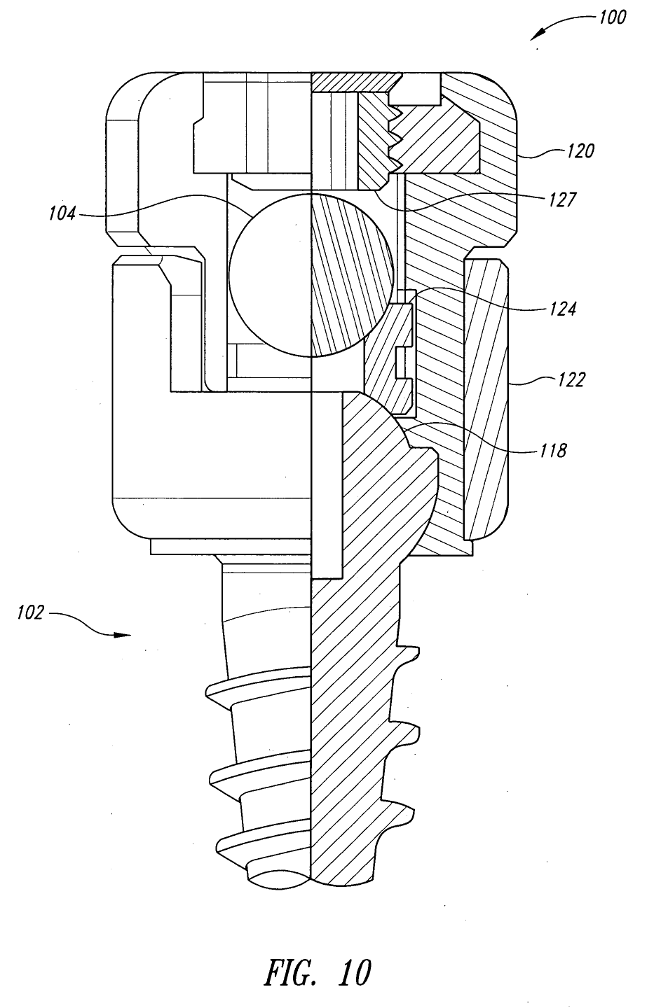

FIGS. 7A, 7B, and 7C show the cap 126 having cam extensions 168, grooves 170, and internal threads 172. The cam extensions 168 include protuberances 176 and lead radii 178. The protuberances 176 operate as an interlocking feature such that when the cap 126 is coupled to the tulip body 120, the protuberances 176 of the cap 126 interlock with the detents 152 of the tulip body 120 (see FIG. 10). In addition, this interlocking feature permits the protuberances 176 to radially restrain the upper portion 130 of the tulip body 120, which may reduce or eliminate post-operative, outward, radial expansion (i.e., splaying) of the upper portion 130 of the tulip body.

In one embodiment, an effective cam length 180 of the cap 126 is slightly larger than an internal, diametrical distance 182 (FIG. 4B) of the cap-mating groove 150 of the upper portion 130 of the tulip body 120. Thus, a maximum length 180 from one cam extension outer surface 169a to another cam extension outer surface 169b is greater than the internal, diametrical distance 182 between the cap-mating grooves 150 of the tulip body 120. When the cap 126 is installed in this type of embodiment, the effective cam length 180 cams open the opening 144 in the upper portion 130 of the tulip body 120 by at least a small amount. This camming action is projected to the lower portion 128 of the tulip body 120 and operates to cause the spherical bore 134 to clamp and/or lock onto the head portion 110 of the pedicle screw 102. This locking of the tulip body 120 onto the pedicle screw 102 occurs before the rod 104 is fixed to the tulip assembly 106 with the setscrew 127.

Operation/Assembly

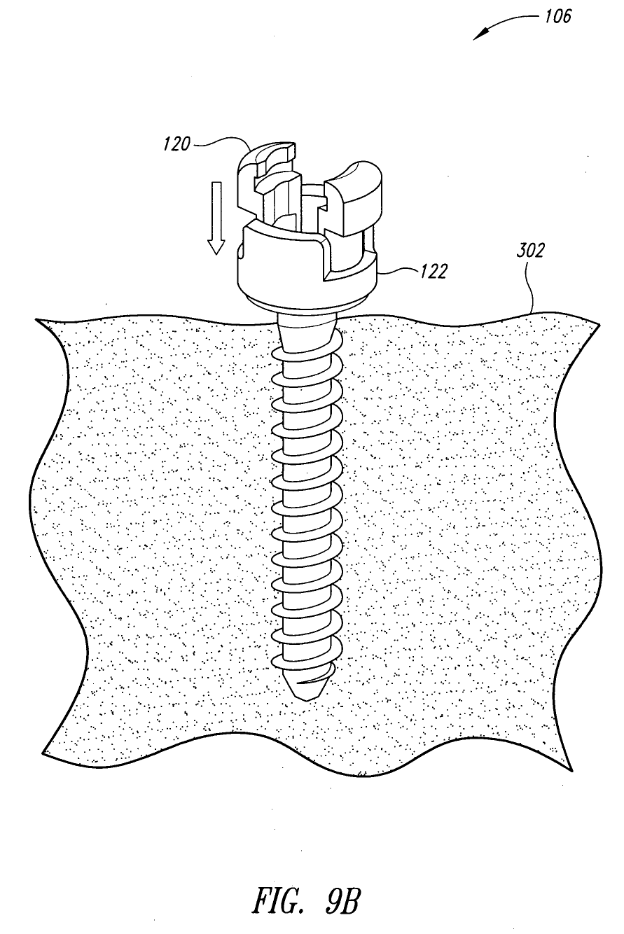

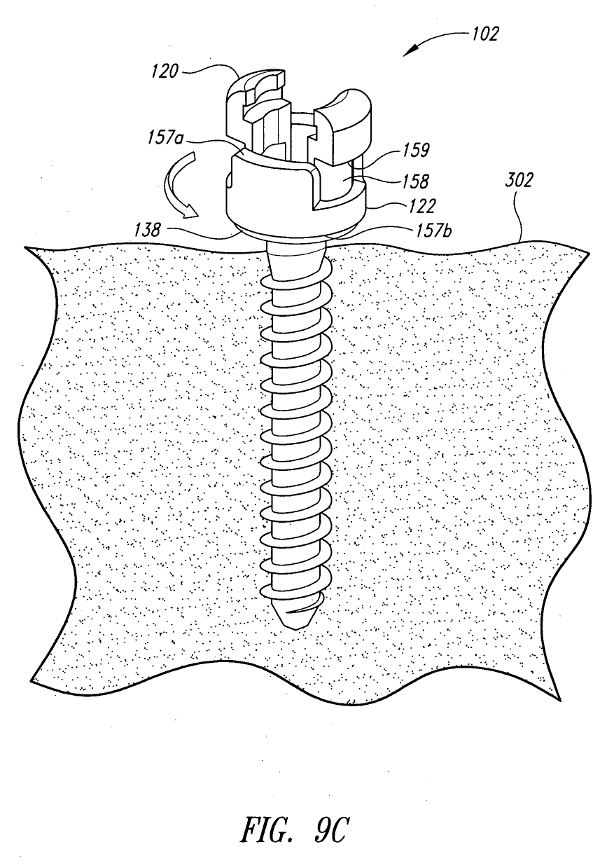

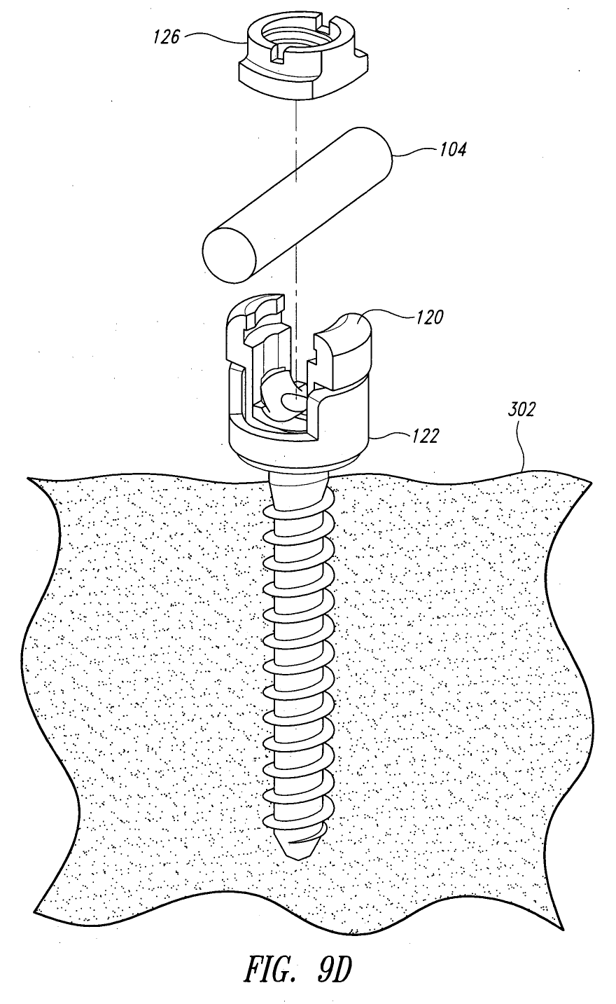

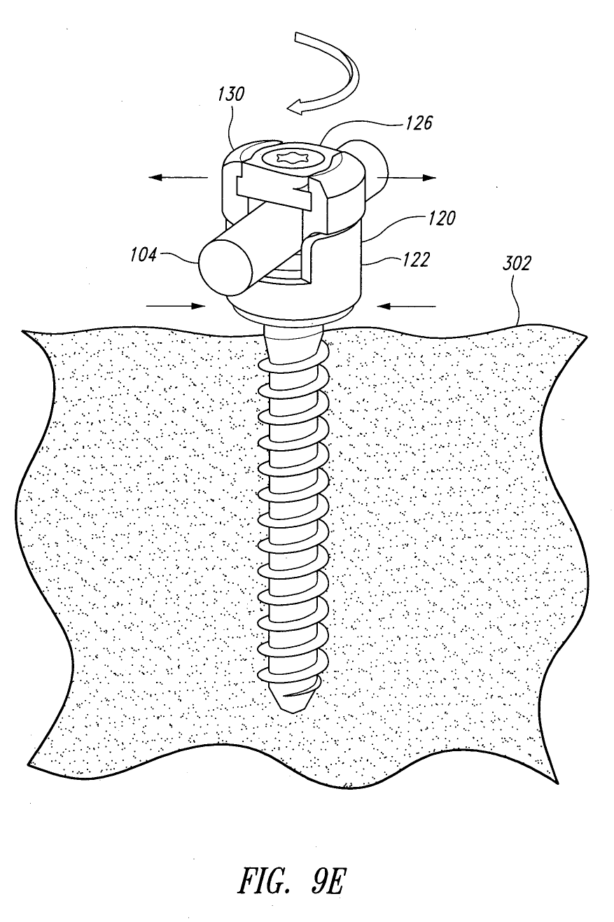

FIG. 8 is a flowchart showing a method 200 of assembling a pedicle screw system 100, according to one illustrated embodiment. In combination and cooperation with method 200, reference may be made to FIGS. 9A-9E to further describe and/or explain aspects of the assembly method 200.

The assembly method 200 begins at step 202 where the collar member 122 and the rod-support member 124 are pre-operatively assembled with the tulip body 120 as described above. It is understood that this pre-operative assembly may take place generally within the hospital or surgical center, possibly even in or near the operating room, or alternatively may take place at a manufacturer before the respective parts are shipped.

As shown in FIG. 9A, the pedicle screw 102 is insertably secured in the bone 302 with the head portion 110 of the pedicle extending above the bone surface 304 in step 204. The upper portion 130 of the tulip body 120 is compressed and/or squeezed to allow the head portion 110 of the pedicle screw 102 to be received in the spherical bore 134 (FIG. 4B) in the lower portion 128 of the tulip body 120 in step 206. The upper portion 130 of the tulip body 120 is released in step 208, which allows the tulip body 120 to re-assume its natural or unloaded position. In addition, the greater diameter 116 (FIG. 2B) of the head portion 110 of the pedicle screw is contiguous with the spherical bore 134 of the tulip body 120 while the lesser diameter 118 (FIG. 2B) contacts the rod-support member 124.

As shown in FIG. 9B, the collar member 122, which is already on the tulip body 120, is slid down the tulip body 120 in step 210. Sliding the collar member 122 down the tulip body 120 keeps the region 142 (FIG. 4B) of the tulip body 120 from re-opening and/or spreading apart and thus retains the head portion 110 of the pedicle screw 102 within the spherical bore 134 (FIG. 4B) of the tulip body 120. In this configuration, the tulip assembly 106 is secured to the head portion 110 of the pedicle screw 102, but remains free to rotate relative to the pedicle screw 102.

As shown in FIGS. 9C and 9D, the collar member 122 is rotated by an amount sufficient to align the cutout 158 in the collar member 122 with the rod-support surface 160 of the rod-support member 124 in step 212. As the collar member 122 is rotated, the upper and lower surfaces 157a, 157b of the collar member 122 become vertically constrained by the shoulder 159 and the radial flange 138 of the tulip body 120, respectively.

As shown in FIG. 9D, the rod 104 is placed in the tulip assembly 106 in step 214. The rod 104 is seated on the rod-support surface 160 of the rod-support member 124. At least a portion of the rod 104 extends through and out of one of the cutouts 158 in the collar member 122. The cap 126 is oriented and placed in the upper portion 130 of the tulip body 120 in step 216.

As shown in FIG. 9E, the cap 126 is rotated by an amount to allow the cam extensions 168 of the cap 126 to engage the grooves 150 in the upper portion 130 of the tulip body 120 in step 218. As the cam extensions 168 of the cap 126 engage the grooves 150, the upper portion 130 of the tulip body 120 is forced to expand radially outward because the maximum outer diameter 180 (FIG. 7A) of the cam extensions 168 is larger than the inner diameter 182 (FIG. 4B) of the grooves 150. The radial, outward expansion of the upper portion 130 of the tulip body 120 causes the lower portion 128, in particular the spherical bore 134, to clamp onto the head portion 110 of the pedicle screw 102. The amount of clamping force is sufficient to substantially prevent any relative movement between the tulip assembly 106 and the pedicle screw 102. Thus, the rotation of the cap 126 into the grooves 150 of the tulip body 120 locks the tulip assembly 106 onto the pedicle screw 102.

As best seen in FIG. 10, the setscrew 127 is threaded into the cap 126 in step 220, which completes the assembly of the pedicle screw system 100. The setscrew 127 applies pressure to the rod 104, which clamps the rod 104 between the rod-support member 124 and the setscrew 127. The rod-support member 124 is in contact with the lesser diameter 118 of the head portion 110 of the pedicle screw 102.

Advantages

One possible advantage of the pedicle screw system 100 is that the dual diameter head portion 110 of the pedicle screw 102 allows the rod-support member 124 to sit low in the tulip assembly 106, which reduces the overall height of the tulip assembly 106 or, alternatively stated, reduces how much the tulip assembly 106 extends above the head portion 110 of the pedicle screw. This reduced height may mitigate soft tissue irritation, especially post-operatively.

Yet another possible advantage is that the dual diameter head portion 110 permits the various components of the tulip assembly 106 to remain concentric, which may permit easier movement (e.g., less frictional binding and/or resistance) between the tulip assembly 106 and the head portion 110 of the pedicle screw 102.

Yet another possible advantage is that the cap 126 may be easily rotated in the tulip body 120 to cause the radial, outward expansion of the upper portion 130 of the tulip body 120. The radial, outward expansion provisionally locks the tulip body 106 to the pedicle screw 102 by causing the lower portion 128, in particular the spherical bore 134, to clamp onto the head portion 110 of the pedicle screw 102. Thus, the angular position of the tulip body 106 relative to the pedicle screw 102 may be quickly locked, then unlocked, and then re-locked at a different angular orientation by merely rotating the cap 126. This flexibility allows the surgeon to repetitively and intra-operatively adjust, if necessary, the angular orientation of the tulip assembly 106 relative to the pedicle screw 102 without causing extra stress to the pedicle screw 102 and/or the bone 302.

Yet another possible advantage is that the protuberances 176 of the cap 126 radially restrain the upper portion 130 of the tulip body 120, thus reducing or possibly eliminating any post-operative splaying and/or undesired flexing of the upper portion 130 of the tulip body 120. Accordingly, the post-operative life of the pedicle screw system 100 may be longer when compared to other, conventional pedicle screw constructs, which in turn may reduce or eliminate any follow-up, repair, and/or maintenance-type spinal operation, for example to fix or replace a broken pedicle screw construct.

All of the above U.S. patents, U.S. patent application publications, U.S. patent applications, foreign patents, foreign patent applications, and non-patent publications referred to in this specification and/or listed in the Application Data Sheet, including but not limited to U.S. Provisional Patent Application No. 60/622,107 filed Oct. 25, 2004, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A tulip assembly coupleable to a pedicle screw having a head portion and a threaded shaft, the head portion coupled to the threaded shaft, the tulip assembly comprising:
a tulip body having an intermediate web, an upper portion positioned above the intermediate web, and a lower portion positioned below the intermediate web, the upper and lower portions displaceable in substantially a radial direction relative to the intermediate web, the lower portion radially expandable by an amount to receive the head portion of the pedicle screw; and
a cap engageable with the upper portion of the tulip body to lock the lower portion of the tulip body onto the head portion of the pedicle screw when the tulip body is at a desired angle relative to the pedicle screw.

2. The tulip assembly of claim 1 wherein the tulip assembly receives the head portion of the pedicle screw intra-operatively and after the pedicle screw is secured into bone.

3. The tulip assembly of claim 1, further comprising:
a collar member that is slideably engageable with the tulip body.

4. The tulip assembly of claim 3 wherein the collar member is slideably moveable between a non-constraining position and a constraining position, the non-constraining position permitting the lower portion of the tulip body to receive the head portion of the pedicle screw, the constraining position substantially reducing outward radial displacement of the lower portion of the tulip body as the cap engages the upper portion of the tulip body.

5. The tulip assembly of claim 1, further comprising:
an externally threaded fastening member coupleable to internal threads formed in the cap.

6. The tulip assembly of claim 1 wherein the cap includes cam extensions to engage cap-mating grooves in the upper portion of the tulip body.

7. The tulip assembly of claim 6 wherein a maximum length from one cam extension outer surface to another cam extension outer surface is greater than an internal, diametrical distance between the cap-mating grooves of the tulip body.

8. The tulip assembly of claim 6 wherein the cap includes protuberances to engage with interlocking features of the upper portion of the tulip body.

9. The tulip assembly of claim 1, further comprising:
a rod-support member having a rod-support surface and a bore, the rod-support member positioned substantially within a counterbored region of the tulip body.

10. The tulip assembly of claim 9 wherein the rod-support surface is contoured to achieve a tight fit with the rod.

11. The tulip assembly of claim 9 wherein the bore of the rod-support member is sized and positioned within the tulip body to permit access to the head portion of the pedicle screw by a driving tool.

12. A pedicle screw system comprising:
a pedicle screw having a head portion and a threaded shaft, the head portion coupled to the threaded shaft; and
a tulip assembly comprising a tulip body and a cap, the a tulip body having an intermediate web, an upper portion positioned above the intermediate web, and a lower portion positioned below the intermediate web, the upper and lower portions displaceable in substantially a radial direction relative to the intermediate web, the lower portion radially expandable by an amount to receive the head portion of the pedicle screw, the cap engageable with the upper portion of the tulip body to lock the lower portion of the tulip body onto the head portion of the pedicle screw when the tulip body is at a desired angle relative to the pedicle screw.

13. The pedicle screw system of claim 12 wherein the head portion of the pedicle screw includes a first diameter and a second diameter.

14. The pedicle screw system of claim 13 wherein the first diameter is larger than the second diameter.

15. The pedicle screw system of claim 12, further comprising:
a collar member slideably receivable by an outer portion the tulip body.

16. The pedicle screw system of claim 15 wherein the collar member is slideably moveable between a non-constraining position and a constraining position, the non-constraining position permitting the lower portion of the tulip body to receive the head portion of the pedicle screw, the constraining position substantially reducing outward radial displacement of the lower portion of the tulip body as the cap engages the upper portion of the tulip body.

17. The pedicle screw system of claim 12, further comprising:
an externally threaded fastening member coupleable to internal threads formed in the cap.

18. The pedicle screw system of claim 17 wherein the externally threaded fastening member is a setscrew.

19. The pedicle screw system of claim 12 wherein the cap includes cam extensions to engage cap-mating grooves in the upper portion of the tulip body.

20. The pedicle screw system of claim 19 wherein a maximum length from one cam extension outer surface to another cam extension outer surface is greater than an internal, diametrical distance between the cap-mating grooves of the tulip body.

21. The pedicle screw system of claim 19 wherein the cap includes protuberances to engage with interlocking features of the upper portion of the tulip body.

22. The pedicle screw system of claim 12, further comprising:
a rod-support member having a rod-support surface and a bore, the rod-support member positioned substantially within a counterbored region of the tulip body.

23. The pedicle screw system of claim 22 wherein the rod-support surface is contoured to achieve a tight fit with the rod.

24. The pedicle screw system of claim 22 wherein the bore of the rod-support member is sized and positioned within the tulip body to permit access to the head portion of the pedicle screw.

25. A method for installing a pedicle screw system into bone, the pedicle screw system including a pedicle screw and a tulip assembly, the method comprising:
inserting the pedicle screw into the bone;
coupling the tulip assembly to a head portion of the pedicle screw by radially, outwardly displacing a lower portion of a tulip body by an amount sufficient to receive the head portion of the pedicle screw, wherein coupling the tulip assembly onto the head portion of the pedicle screw occurs after the pedicle screw has been inserted into the bone;
placing at least a section of a rod onto a rod-support member that is positioned within the tulip body;
fixing the tulip assembly to the head portion of the pedicle screw by rotationally engaging a cap with the upper portion of the tulip body, the cap configured to cam the upper portion of the tulip body radially outward when rotated, and in turn, cause the lower portion of the tulip body to clamp onto the head portion of the pedicle screw; and
inserting a fastening member into the tulip assembly to provide a downward force on at least a portion of the section of the rod to fixedly retain the same in the tulip assembly.

26. The method of claim 25 wherein radially, outwardly displacing the lower portion of the tulip body is caused by squeezing the upper portion of the tulip body.

27. The method of claim 25 wherein radially, outwardly displacing the lower portion of the tulip assembly by the amount sufficient to receive the head portion of the pedicle screw includes radially, outwardly displacing the lower portion of the tulip assembly to guide the tulip assembly over a maximum diameter section of the head portion of the pedicle screw.

28. The method of claim 25 wherein coupling the tulip assembly to a head portion of the pedicle screw is accomplished intra-operatively.

29. The method of claim 25 wherein inserting the fastening member into the tulip assembly includes inserting a setscrew into the cap.
